SEQUENCE LISTING (12 sequences)

SEQ ID NO: 1 - DNA, 21 bp, Artificial Sequence
  Forward primer for Tlr9
gagaatcctccatctcccaac

SEQ ID NO: 2 - DNA, 19 bp, Artificial Sequence
  Reverse primer for Tlr9
ccagagtctcagccagcac

SEQ ID NO: 3 - DNA, 20 bp, Artificial Sequence
  Forward primer for Zbp1
caggaaggccaagacatagc

SEQ ID NO: 4 - DNA, 21 bp, Artificial Sequence
  Reverse primer for Zbp1
gacaaataatcgcaggggact

SEQ ID NO: 5 - DNA, 22 bp, Artificial Sequence
  Forward primer for Ifi204
tgcgttttgtgaagaagtacca

SEQ ID NO: 6 - DNA, 20 bp, Artificial Sequence
  Reverse primer for Ifi204
ggacctgcttcttgaccatt

SEQ ID NO: 7 - DNA, 20 bp, Artificial Sequence
  Forward primer fo Ifng
atctggaggaactggcaaaa

SEQ ID NO: 8 - DNA, 26 bp, Artificial Sequence
  Reverse primer for Ifng
ttcaagacttcaaagagtctgaggta

SEQ ID NO: 9 - DNA, 18 bp, Artificial Sequence
  Forward primer for Tlr3
ccaccagcgagagcactt

SEQ ID NO: 10 - DNA, 20 bp, Artificial Sequence
  Reverse primer for Tlr3
aaagatcgagctgggtgaga

SEQ ID NO: 11 - DNA, 21 bp, Artificial Sequence
  Forward primer for Irf7
cttcagcactttcttccgaga

SEQ ID NO: 12 - DNA, 20 bp, Artificial Sequence
  Reverse primer for Irf7
tgtagtgtggtgacccttgc

(19) United States
(12) Patent Application Publication
(10) Pub. No.: US 2021/0386804 A1
(43) Pub. Date: Dec. 16, 2021

(54) Combination of viral superinfection therapy with subthreshold doses of nivolumab plus ipilimumab in chronic HBV patients

(71) Applicant: Tibor BAKÁCS, Budapest (HU)

(72) Inventor: Tibor BAKÁCS, Budapest (HU)

(21) Appl. No.: 17/343,851

(22) Filed: Jun. 10, 2021

Related U.S. Application Data
(60) Provisional application No. 63/037,614, filed on Jun. 11, 2020.

(51) Int. Cl. A61K 35/76; C07K 16/28

(52) U.S. Cl. CPC A61K 35/76; A61K 2039/545; C07K 16/2818

(57) ABSTRACT

The present invention relates to the combination of attenuated non-pathogenic avian double-stranded (ds) RNA viral vector (IBDV) with an anti-PD-1 antibody and/or an anti-CTLA-4 antibody for treating Hepatitis B virus (HBV) infection.

This application claims priority to provisional application Ser. No. 63/037,614, filed Jun. 11, 2020, the entire disclosure of which is hereby incorporated by reference herein.

### BACKGROUND OF THE INVENTION

Hepatitis B virus (HBV) is a major global public health threat. Worldwide more than 257 million people are chronically infected and over 887 000 deaths are caused by HBV every year¹. In most cases, nucleoside analog HBV polymerase inhibitors (NUCs) treatment must continue for life because they usually suppress rather than cure infection. Importantly, current therapies reduce but do not eliminate the risk of hepatocellular carcinoma (HCC)². Therefore, up to 80 million people will die from HCC. A compounding problem is that an unknown number of the two billion people alive today who resolved HBV infection earlier but maintain a viral covalently closed circular DNA (cccDNA) reservoir in their liver. If such people are exposed to therapies inducing immune suppression, HBV infection can be reactivated. A good example for this is the direct acting antiviral (DAA) drug treatment of hepatitis C virus (HCV) infected patients who have silent HBV coinfection. Reactivation of HBV in these patients is a recently identified safety concern. The FDA issued a black box warning that all HCV patients who are going to be treated with DAA agents must first undergo HBV panel testing³.

Long-term use of NUCs is associated with toxicity, noncompliance, viral resistance, and unsustainable cost implications for many of the most heavily affected countries⁴. Peginterferon-a (PEG-IFN-α) treatment, unlike NUCs, does not result in antiviral resistance and is administered for a finite period. However, PEG-IFN-α is associated with many troublesome, occasionally with serious, even life-threatening side effects. Discontinuation and dose modification have been reported in 6%-9% and 31%-47% of patients, respectively, treated in the PEG-IFN-α registration trials for HBV^{5 6 2}.

The evolving research landscape in host directed therapies (HDT) of infectious diseases for the development of broad-spectrum antivirals^{8 9} and new results concerning the exploitation of safe subthreshold doses of immune checkpoint inhibitors (IC's) for the treatment of advanced cancer¹⁰ are creating unique opportunities to translate science into new therapies with the potential to substantially improve the lives of many million patients with chronic hepatitis B (CHB) infections. Outlined in this Viewpoint is the unmet need in CHB, particularly concerning the functional cure of HBV and design a trial to address the real challenges. According to our hypothesis, functional cure of CHB could be achieved with the combination of two clinically tested though not registered therapies: 1) the viral superinfection therapy (SIT), which is a HDT, for the reduction of HBV load, and 2) immune stimulation by subthreshold doses of nivolumab (0.5 mg/kg) plus ipilimumab (0.3 mg/kg), which was demonstrated to be safe and effective in advanced cancer¹⁰. Gane et al., recently demonstrated in virally suppressed HBeAg-negative patients that a subthreshold dose (0.3 mg/kg) nivolumab blockade was well-tolerated and led to HBsAg decline in most patients¹¹. A proof-of-principle CHB trial is proposed as a model for next generation anti-HBV therapy. The concept and the protocol are open for interactive discussion.

According to the International Coalition to Eliminate HBV (ICE-HBV) Functional Cure of Chronic HBV During Finite Treatment Course is a Realistic Goal

The current goal of HBV therapy is a partial cure; the new realistic goal should be a functional cure; while complete (sterilizing) cure with undetectable HBsAg and complete eradication of HBV DNA including cccDNA minichromosome reservoir or integrated HBV DNA from every infected cell will hopefully be achieved in the future. According to The International Coalition to Eliminate HBV (ICE-HBV) reducing a patient's risk of death due to liver disease to that of a person with a resolved infection is a feasible therapeutic goal ¹. In fact, functional cure with sustained undetectable HBsAg and HBV DNA in serum with or without seroconversion, with persistently low amounts of intrahepatic cccDNA and HBV DNA integration seems to be achievable in the near future provided it addresses high viral burden and weak immune response, respectively. Optimally, therapy should eliminate HbsAg, HBV DNA and should also trigger a durable effect by stimulating HBV-specific host immune responses that mimic spontaneous resolution of HBV infection. This is based on the fact that natural immune responses can control HBV in more than ninety percent of those infected as adults. Since the humanitarian, public health and financial burdens of chronic hepatitis B are enormous there is strong motivation to find a cure. For global elimination of HBV, these new therapies will need to be safe, convenient and affordable in low-income countries with the highest burden of HBV infection¹².

Functional Cure of Chronic HBV Infection can Only be Achieved by Combination of Antiviral and Immunomodulatory Therapies

A number of drugs are being evaluated but none of them is capable of curing HBV infection on their own. The high HBV burden should be reduced with novel antiviral approaches, and then the innate and adaptive host immune responses against HBV should be restored with immunomodulatory agents. Several promising candidates are already in Phase II trials². For the reduction of high viral burden capsid allosteric modulators (CAPs), transcription inhibitors, gene editing, RNA interference, and nucleic acid polymers (NAPs) are developed. Weak immune response is manipulated by stimulation of antiviral effector cells, generating new T cells and “rescuing” exhausted T cells. The latter is required because continuous antigen stimulation impairs (exhaust) progressively accumulating HBV-specific T cells in the inflamed liver, in which 40%-100% of the 10¹¹ hepatocytes are infected expressing HBV-specific antigens, and the viral load is also high. Under these conditions, T cells have an increased propensity to express the coinhibitory receptors, which correlates with viral load. Here we propose the sequential administration of an antiviral and an immunomodulatory therapy both of which have been clinically tested though not registered yet. First, the viral burden should be reduced by the viral superinfection therapy (SIT), a HDT approach⁹, and then HBV specific T cell response should be restored by a safe subthreshold dose of nivolumab plus ipilimumab blockade¹⁰ as described below.

SIT has been Validated in HBV and HCV Patients, Respectively

SIT is a HDT, which has been previously validated in patients infected with two completely different viruses, the hepatitis B (DNA), and hepatitis C (RNA) viruses^{9 13 14}. The proof of SIT concept was demonstrated in acute B or C viral hepatitis patients in a preliminary Phase II clinical trial¹⁵. Significantly less patients progressed into chronic disease and suffered from relapse in the virus treated groups, while remission within one month of treatment was more frequent in the virus treated groups. In contrast, late remissions (requiring more than 6 months) were recorded significantly more frequently in the control groups. The duration of the first icteric phase was also shortened by the IBDV superinfection treatment. No serious treatment-related adverse events were observed. Importantly, the orally administered SIT was safe and effective even in in parenchymally decompensated hepatitis B and C patients with various life-threatening complications (e.g. portal hypertension, diuretic-resistant ascites, progressive jaundice, generalized edema, hepatic encephalopathy)¹⁶.

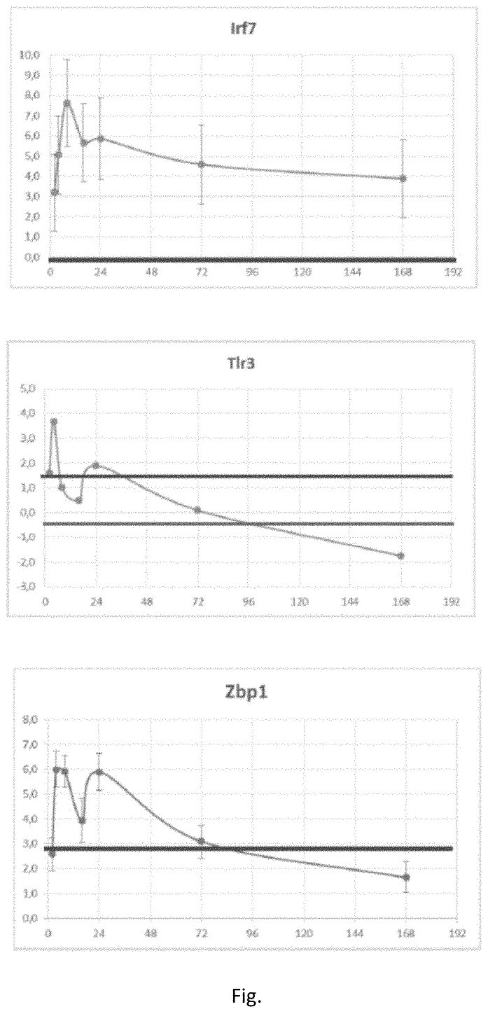

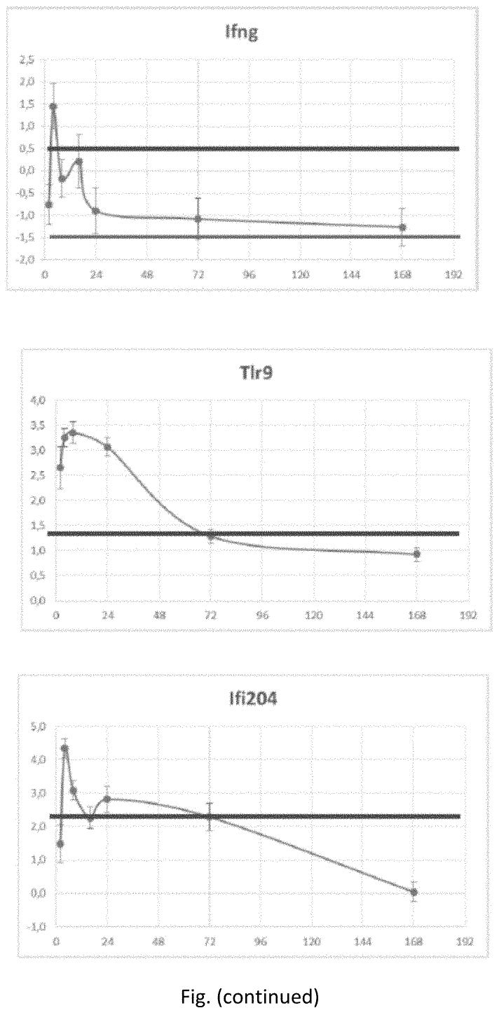

SIT exerts post-infection interference via the constant presence of an attenuated non-pathogenic avian double-stranded (ds) RNA viral vector (IBDV) which boosts the endogenous innate IFN response via toll-like receptors (TLRs). IBDV is administered orally continuously usually for 24 weeks but up to 52 weeks in severe cases. Importantly, no serious side effects were observed during IBDV superinfection therapy. In sharp contrast, systemic IFN-therapy is associated with a wide array of severe adverse effects. One possible explanation for the different safety of SIT and systemic IFN therapy could be the dissimilar target range of the two therapies. Type I and II IFN receptors are found on the surface of most cell types such that systemic IFN therapy has an ubiquitous nature of signaling¹⁷. Viruses, in contrast, have very restricted cellular and host tropism¹⁸. Consistent with this, IBDV interacts with appropriate cells such that its dsRNA is recognized by specific receptors (e.g. TLR3). These activate several gene families from within. This way, expression levels of IFN-related genes such as for example the toll-like receptor 9 (Tlr9), Z-DNA binding protein 1 (Zbp1), interferon activated gene 204 (Ifi204), interferon gamma (IFN-γ), toll-like receptor 3 (Tlr3), interferon regulatory factor 7 (Irf7) genes are increased even after a single intravenous injection of IBDV (R903/78) drug candidate, as depicted in FIG. 1. This is a major difference between systemic IFN-based and superinfection therapy. Regardless of the specific mechanism of action, it is already clear that the systemic and virus-based therapeutic modalities are different. We hypothesize that induction of several innate immune system gene families by the dsRNA of IBDV is capable counteracting immune surveillance evasion more effectively than systemic IFN therapy can do. Future clinical studies need to evaluate HBV integration status and correlate with the efficacy of treatment. SIT is essentially a presystemic interferon therapy without induction of systemic IFN-α which is associated with severe side effects. However, unlike RNA interference or CRISPR/Cas9 technologies, SIT has durable off-treatment effects while it has no off-target safety issues.

Autoimmunity is Emerging as the “Achilles' Heel” of Immunotherapy Due to Safety Issues of ICIs

The remarkable success of immune checkpoint inhibitor (ICI) therapies in cancer could offer a promising new complementary strategy to achieve the functional cure of HBV ^{11 2 19 20}. The problem is that cancer regression can only be achieved with ICI therapies by paying a price—tolerance to healthy self-tissues is compromised. Science has acknowledged that the patients treated by ICI drugs are “human experiments” of the autoimmune process²¹, while autoimmunity is emerging as the “Achilles' heel” of immunotherapy²². Since most of the safety and efficacy profiles of ICI agents were established in oncology patients, the extent to which those parameters are appropriate for CHB patients remains to be determined. In order to administer ICI therapies safely and effectively in CHB patients, it is important to consider the safety issue of ICI therapy in advanced cancer (see a detailed review in ¹⁰).

While ICI monotherapies provided a durable benefit to only a minority of patients (15-20%), combination of 2 different ICIs improved response in about two-third of patients. For example, the 3-year overall survival (OS) rate reached 63% in stage III or IV melanoma who received concurrent ipilimumab and nivolumab now approved for patients with unresectable or metastatic melanoma²³. This spectacular result was, however, associated with irAEs (any-grade) in 96.8% of patients, but grade 3 and 4 in 58.5% of patients leading to discontinuation in 24.5%. A comprehensive meta-analysis of 23 clinical trials of nivolumab plus ipilimumab in advanced malignancies including 2,114 and 2,674 patients eligible for efficacy and safety analysis, respectively showed objective response rate (ORR) in 34.5% of patients, which was associated with grade 3-4 irAEs in 39.9% of patients, while treatment-related death occurred in 2.0% of patients²⁴.

With the benefit of hindsight, our prediction in 2012 concerning the safety of ICI therapy seems to be validated²⁵. Then, we emphasized that the widespread, dose-dependent irAEs of ipilimumab could have been expected. This view was based on our theory that all T cells possess physiologic self-reactivity^{26 27}. Indeed, we argued for a profound theoretical point against the consensus of experts. Since the anti-CTLA-4 immune checkpoint blockade cannot be restricted to the targeted tumor-specific T cell population, such that this blockade induces an uncontrolled pan T cell activation, tolerance to healthy self-tissues will be compromised. Therefore, we hypothesized that the anti-CTLA-4 therapy may have mechanisms similar to that occurring in inherited human CTLA4 haplo-insufficiency²⁸. To resolve the safety issue of ICI therapies a therapeutic paradigm shift is required. Instead of trying to put the genie back in the bottle by immune suppressive treatments, we should harness the autoimmune forces for antitumor effects. To this end, Slavin et al. proposed first that a finely tuned, subthreshold ipilimumab dose (0.3 mg/kg) would induce a prolonged auto-graft-versus-host-disease (auto-GVHD) that would improve the antitumor efficacy of the patients' own lymphocytes in minimal residual disease (MRD)²⁹. In this way, the same goal could be achieved by an antibody (ipilimumab) in analogy the adoptive transfer of alloreactive donor lymphocytes, but of course, without the risk of GVHD³⁰.

Subthreshold Doses of Ipilimumab Plus Nivolumab Combined with Hyperthermia and IL-2 was Safe and Effective in 131 Unselected Advanced Metastatic Cancers

Based on the theory of Slavin et al., Kleef et al. developed a combination therapy consisting of different T cell stimulation modalities such as hyperthermia and interleukin-2, which were supplemented with subthreshold doses of nivolumab (0.5 mg/kg) plus ipilimumab (0.3 mg/kg). The rationale for subthreshold ICI doses is the quantitative paradigm of T cell stimulation which states that T cell activation is the outcome of signals from the TCR, co-stimulatory/co-inhibitory receptors and cytokines added together^{31,32} Since the individual (including subthreshold) stimulating effects add up, they are able to achieve the magnitude of T cell stimulation required for tumor eradication. The proof-of-concept of the combination therapy was demonstrated in stage IV cancer patients, who had exhausted all conventional treatment^{33 34}. The preliminary results were then confirmed by a retrospective analysis of 131 stage IV patients with 23 different types of cancer. The ORR was found to be 31,3%, progression free survival (PFS) was 10 months, survival-probability at 12 months was 66.5%. However, subthreshold ICI doses were associated with irAEs of grade 3 and 4 in only 6.11% and 2.29% of patients, respectively (Kleef et al., manuscript in preparation). The rationale for using subthreshold ICI doses have been supported by several studies demonstrating that despite a dose-dependent increase in irAEs, no improvement in PFS, OS, or disease control rate (DCR) were identified with escalating doses of ICIs. In fact, lower doses may reduce toxicity and cost without compromising disease control or survival^{35 36}. Such a way, the lessons learnt from the safe subthreshold nivolumab plus ipilimumab therapy of advanced cancer patients could be exploited for the immune stimulatory treatment of CHB patients as described below.

### DESCRIPTION OF THE INVENTION

Testable hypothesis based on the aforementioned information: combination of SIT with sequential subthreshold nivolumab plus ipilimumab therapy is likely to provide synergistic activation of the immune system toward induction of therapeutic anti-HBV responses aiming to accomplish functional cure of CHB during a finite treatment course

Continuous antigen stimulation in CHB exhausts HBV-specific T cells because insufficient costimulatory signals are outweighed by an excess of coinhibitory signals. HBV-specific T cell response can, however, be restored by blockading the coinhibitory CTLA-4 and PD-1 receptors, respectively, which have nonredundant complementary roles in the attempt to reconstitute an effective HBV-specific T cell response³⁷. This way, hopefully, infected hepatocytes will be eliminated by HBV-specific T cells, while cccDNA will be eliminated by non-cytolytic mechanisms, and reinfection of newly generated hepatocytes will be prevented by clearance of infective HBV by HBV-specific antibodies. Since the clinical use of an immune checkpoint blockade may be limited by potential side effects, the possible clinical benefits and the risks of ICI agents in CHB patients will have to be balanced by close and careful monitoring. These are mainly related to irAEs induced by ICIs, as well as to increased liver inflammation, that might lead to hepatitis exacerbation, as reported in HBsAg-positive cancer patients³⁷. The good news however is that Gane et al., recently demonstrated in virally suppressed HBeAg-negative patients that a subthreshold dose (0.3 mg/kg) nivolumab blockade was well-tolerated and led to HBsAg decline in most patients and sustained HBsAg loss in 1 patient¹¹. The examination of combinatorial strategies for treatment of CHB is encouraged².

Therefore, a combination is proposed which consists of two clinically tested therapies: 1) SIT that will reduce the high HBV burden; and 2) a sequential subthreshold nivolumab plus ipilimumab therapy will trigger a durable downstream effect by restoring the adaptive host immune responses against HBV. Eventually, this could result in an immune-mediated destruction and/or non-cytolytic elimination of HBV from infected cells. Since SIT and subthreshold nivolumab plus ipilimumab treatment activate different arms of the immune system, they may achieve synergistic controlling effects of CHB infection.

Proposed Proof-of-Principle Trial in Virally Suppressed Patients with (HBeAg)-Negative CHB

R903/78 product containing 5×10⁶ Infectious Units (IU) of IBDV will be administered orally; the patients will be treated with the investigational product daily for 24 weeks; then an off-label very low-dose ICI combination treatment (nivolumab [0.5 mg/kg] plus ipilimumab [0.3 mg/kg]) will be administered. Patients will receive nivolumab IV over 60 minutes on days 1, 15, and 29 and ipilimumab IV over 90 minutes on day 1 and day 15.

The combination of SIT and subthreshold ICI will be carried out in two sequential steps; step 1, studying the safety of SIT by administration of the new viral drug candidate R903/78 alone. Step 2, studying the safety of combined administration of the new viral drug candidate R903/78 and low-dose checkpoint inhibitors.

Primary objective: The primary objective is to determine the safety profile of the R903/78 product used for elimination of HBV infection in patients with CHB.

Secondary objectives: The secondary objective is to determine the efficacy of the R903/78 product in eliminating HBV infection of patients with CHB.

To determine the effect of R903/78 on each of the following factors:

i. quantitative value of HBsAg

ii. circulating viral DNA (HBV PCR)

iii. HBV RNA

iv. HBV specific CD8 T cells

v. serum transaminases levels

To determine the safety of R903/78 by assessing adverse events and safety laboratory factors. CHB patients will be recruited into a single-center study.

Number of participants: 40 patients.

Inclusion Criteria:

Adult CHB patients (18-70 years) documented by HBsAg+ve at least 6 months before screening, with low viremia <2000 IU/mL (naturally or achieved by ongoing NUCs), HBeAg+ve & HBeAg-ve will be included 1:2, non-cirrhotic patients.

Exclusion Criteria:

Evidence of competing liver etiology other than CHB (NAFLD & Gelbert are optional if investigators assess them non active), HDV+ve by RNA testing, HCV or HIV coinfection, evidence of any malignancy (including HCC), pregnancy or pregnancy potential, any immunosuppression, patients underwent transplantation (hematopoietic or solid organ), patients with cirrhosis and/or portal hypertension.

### BRIEF DESCRIPTION OF THE FIGURES

FIGURE. Analysis of expression levels of virus-activated genes following intravenous injection of IBDV (R903/78) drug candidate. X=delta-delta Ct values presented as log 2 values; Y=time after IV injection of IBDV (R903/78) (hrs.)

At 0 minute the mice were inoculated with the 1 million IBDV particles in PBS intravenously using tail vein. At appropriate time (2 h, 4 h, 8 h, 16 h, 24 h, 72 h, 1 week) the mice were sacrificed by CO2 asphyxiation and the liver was isolated for RNA purification. Total RNAs were isolated as described previously³⁸. Quantitative realtime PCR (QRT-PCR) analysis was performed as described previously³⁹. Relative expression ratios were calculated as normalized ratios to mouse GUSB gene. Each sample was tested in triplicate. The final relative gene expression ratios were calculated as delta-delta Ct values and presented as log 2 values. For expression analysis virus-activated gene primers were designed using the online Roche Universal Probe Library (UPL) Assay Design Center.

The quality of the primers was verified by MS analysis provided by Bioneer (Daejeon, Korea). Table 1 presents the sequence information about the UPL probes and primers.

TABLE 1

| | Accession | | | UPL |
|---|---|---|---|---|
| Gene name | no. | Forward primer | Reverse primer | probe |
| toll-like receptor 9 (Tlr9) | NM_031178 | gagaatcctccatctcccaac | ccagagtctcagccagcac | #79 |
| | | (SEQ ID NO: 1) | (SEQ ID NO: 12) | |
| Z-DNA binding protein 1 | NM_021394 | caggaaggccaagacatagc | gacaaataatcgcaggggact | #109 |
| (Zbp1) | | (SEQ ID NO: 3) | (SEQ ID NO: 4) | |
| interferon activated | NM_008329 | tgcgttttgtgaagaagtacca | ggacctgcttcttgaccatt | #2 |
| gene 204 (Ifi204) | | (SEQ ID NO: 5) | (SEQ ID NO: 6) | |
| interferon gamma (Ifng) | BC119063 | atctggaggaactggcaaaa | ttcaagacttcaaagagtctgaggta | #21 |
| | | (SEQ ID NO: 7) | (SEQ ID NO: 8) | |
| toll-like receptor 3 (Tlr3) | AF355152 | ccaccagcgagagcactt | aaagatcgagctgggtgaga | #26 |
| | | (SEQ ID NO: 9) | (SEQ ID NO: 10) | |
| interferon regulatory | BC138799 | cttcagcactttcttccgaga | tgtagtgtggtgacccttgc | #25 |
| factor 7 (Irf7) | | (SEQ ID NO: 11) | (SEQ ID NO: 12) | |

### REFERENCES

1. Revill P A, Chisari F V, Block J M, et al. A global scientific strategy to cure hepatitis B. Lancet Gastroenterol Hepatol. 2019; 4(7):545-558.
2. Gane E. Ongoing clinical trials with novel drugs to cure HBV and HDV infections. 6th ANRS HBV Cure Workshop; 2019; Paris.
3. Bersoff-Matcha Si, Cao K, Jason M, et al. Hepatitis B Virus Reactivation Associated With Direct-Acting Antiviral Therapy for Chronic Hepatitis C Virus: A Review of Cases Reported to the U.S. Food and Drug Administration Adverse Event Reporting System. Ann Intern Med. 2017; 166(11):792-798.
4. Papatheodoridis G V, Manolakopoulos S, Dusheiko G, Archimandritis Therapeutic strategies in the management of patients with chronic hepatitis B virus infection. The Lancet Infectious Diseases. 2008; 8(3):167-178.
5. Janssen HLA, van Zonneveld M, Senturk H, et al. Pegylated interferon alfa-2b alone or in combination with lamivudine for HBeAg-positive chronic hepatitis B: a randomised trial. The Lancet. 2005; 365(9454):123-129.
6. Lau G K, Piratvisuth T, Luo K X, et al. Peginterferon Alfa-2a, lamivudine, and the combination for HBeAg-positive chronic hepatitis B. N Engl J Med. 2005; 352(26):2682-2695.
7. Marcellin P, Lau G K, Bonino F, et al. Peginterferon alfa-2a alone, lamivudine alone, and the two in combination in patients with HBeAg-negative chronic hepatitis B. N Engl J Med. 2004; 351(12):1206-1217.
8. Kaufmann SHE, Dorhoi A, Hotchkiss R S, Bartenschlager R. Host-directed therapies for bacterial and viral infections. Nat Rev Drug Discov. 2018; 17(1):35-56.
9. Kovesdi I, Bakacs T. Therapeutic exploitation of viral interference. Infectious Disorders—Drug Targets. 2019; 19:1-1.
10. Bakacs T, Moss R W, Kleef R, Szasz M A, Anderson C C. Exploiting autoimmunity unleashed by low-dose immune checkpoint blockade to treat advanced cancer. Scand J Immunol. 2019:e12821.
11. Gane E, Verdon D J, Brooks A E, et al. Anti-PD-1 blockade with nivolumab with and without therapeutic vaccination for virally suppressed chronic hepatitis B: A pilot study. J Hepatol. 2019; 71(5):900-907.
12. Block T M, Alter H, Brown N, et al. Research priorities for the discovery of a cure for chronic hepatitis B: Report of a workshop. Antiviral Res. 2018; 150:93-100.
13. Bakacs T, Safadi R, Kovesdi I. Post-infection viral superinfection technology could treat HBV and HCV patients with unmet needs. Hepatol Med Policy. 2018; 3:2.
14. Hornyak A, Lipinski K S, Bakonyi T, et al. Effective multiple oral administration of reverse genetics engineered infectious bursal disease virus in mice in the presence of neutralizing antibodies. J Gene Med. 2015; 17(6-7):116-131.
15. Csatary L K, Telegdy L, Gergely P, Bodey B, Bakacs T. Preliminary report of a controlled trial of MTH-68/B virus vaccine treatment in acute B and C hepatitis: a phase II study. Anticancer Res. 1998; 18(2B):1279-1282.
16. Csatary L K, Schnabel R, Bakacs T. Successful treatment of decompensated chronic viral hepatitis by bursal disease virus vaccine. Anticancer Res. 1999; 19(1B):629-633.
17. de Weerd N A, Nguyen T. The interferons and their receptors-distribution and regulation. Immunol Cell Biol. 2012; 90(5):483-491.
18. Nomaguchi M, Fujita M, Miyazaki Y, Adachi A. Viral tropism. Front Microbiol. 2012; 3:281.
19. Gane E J. Future anti-HBV strategies. Liver Int. 2017; 37 Suppl 1:40-44.
20. Pham E A, Perumpail R B, Fram al, Glenn J S, Ahmed A, Gish R G. Future Therapy for Hepatitis B Virus: Role of Immunomodulators. Curr Hepatol Rep. 2016; 15(4):237-244.
21. Couzin-Frankel J. Autoimmune diseases surface after cancer treatment. Science. 2017; 358(6365):852.
22. June C H, Warshauer J T, Bluestone J A. Is autoimmunity the Achilles' heel of cancer immunotherapy? Nat Med. 2017; 23(5):540-547.
23. Callahan M K, Kluger H, Postow M A, et al. Nivolumab Plus Ipilimumab in Patients With Advanced Melanoma: Updated Survival, Response, and Safety Data in a Phase I Dose-Escalation Study. J Clin Oncol. 2018; 36(4):391-398.
24. Xu H, Tan P, Ai J, et al. Antitumor Activity and Treatment-Related Toxicity Associated With Nivolumab Plus Ipilimumab in Advanced Malignancies: A Systematic Review and Meta-Analysis. Front Pharmacol. 2019; 10:1300.
25. Bakacs T, Mehrishi J N, Moss R W. Ipilimumab (Yervoy) and the TGN1412 catastrophe. Immunobiology. 2012; 217(6):583-589.
26. Bakacs T, Mehrishi J N, Szabados T, Varga L, Szabo M, Tusnady G. T cells survey the stability of the self: a testable hypothesis on the homeostatic role of TCR-MHC interactions. Int Arch Allergy Immunol. 2007; 144(2):171-182.
27. Szabados T, Bakacs T. Sufficient to recognize self to attack non-self: Blueprint for a one-signal T cell model. Journal of Biological Systems. 2011; 19(2):299-317.
28. Bakacs T, Mehrishi J N. Anti-CTLA-4 therapy may have mechanisms similar to those occurring in inherited human CTLA4 haploinsufficiency. Immunobiology. 2014; 220:624-625.
29. Slavin S, Moss R W, Bakacs T. Control of minimal residual cancer by low dose ipilimumab activating autologous anti-tumor immunity. Pharmacol Res. 2014; 79:9-12.
30. Bakacs T, Moss, R. W., Kleef, R., Szasz, M. A., Andersone, C. C. Exploiting autoimmunity unleashed by low-dose immune checkpoint blockade to treat advanced cancer. ScandJImmunol in press. 2019; X(Y).
31. Gett A V, Hodgkin P D. A cellular calculus for signal integration by T cells. Nat Immunol. 2000; 1(3):239-244.
32. Marchingo J M, Kan A, Sutherland R M, et al. T cell signaling. Antigen affinity, costimulation, and cytokine inputs sum linearly to amplify T cell expansion. Science. 2014; 346(6213):1123-1127.
33. Kleef R, Moss R W, Szasz A M, Bohdjalian A, Bojar H, Bakacs T. From Partial to Nearly Complete Remissions in Stage I V Cancer Administering Off-label Low-Dose Immune Checkpoint Blockade in Combination with High Dose Interleukin-2 and Fever Range Whole Body Hyperthermia. ASCO; 2016; Chicago, USA.
34. Kleef R, Moss R, Szasz A M, Bohdjalian A, Bojar H, Bakacs T. Complete Clinical Remission of Stage I V Triple-Negative Breast Cancer Lung Metastasis Administering Low-Dose Immune Checkpoint Blockade in Combination With Hyperthermia and Interleukin-2. Integr Cancer Ther. 2018; 17(4):1297-1303.
35. Sen S, Hess K R, Hong D S, et al. Impact of immune checkpoint inhibitor dose on toxicity, response rate, and survival: A pooled analysis of dose escalation phase 1 trials. J Clin Oncol. 2018; 36(15_suppl):3077-3077.
36. Renner A, Burotto M, Rojas C. Immune Checkpoint Inhibitor Dosing: Can We Go Lower Without Compromising Clinical Efficacy? J Glob Oncol. 2019; 5:1-5.
37. Boni C, Barili V, Acerbi G, et al. HBV Immune-Therapy: From Molecular Mechanisms to Clinical Applications. Int J Mol Sci. 2019; 20(11).
38. Kalman J, Palotas A, Juhasz A, et al. Impact of venlafaxine on gene expression profile in lymphocytes of the elderly with major depression-evolution of antidepressants and the role of the “neuro-immune” system. Neurochem Res. 2005; 30(11):1429-1438.
39. Nagy L I, Molnar E, Kanizsai I, et al. Lipid droplet binding thalidomide analogs activate endoplasmic reticulum stress and suppress hepatocellular carcinoma in a chemically induced transgenic mouse model. Lipids Health Dis. 2013; 12:175.

1. A combination of attenuated non-pathogenic avian double-stranded (ds) RNA viral vector (IBDV) with an anti-PD-1 antibody and/or an anti-CTLA-4 antibody for use in the treatment of Hepatitis B virus (HBV) infection.

2. The combination according to claim 1, wherein the viral vector is R903/78.

3. A method for the treatment of HBV infection comprising the administration of a combination defined in claim 1, wherein the administration includes 0.5 mg/kg or lower dose of anti-PD-1 antibody with co-administration of 0.3 mg/kg or lower dose of anti-CTLA-4 antibody.

4. The combination for use or method according to claim 1, wherein the anti-PD-1 antibody and the anti-CTLA-4 antibody are administered simultaneously or sequentially.

5. The combination for use or method according to claim 1, wherein the anti-PD-1 antibody and the anti-CTLA-4 antibody are administered at different times.

6. The combination for use or method according to claim 1, wherein the anti-PD-1 antibody is nivolumab or pembrolizumab and the anti-CTLA-4 antibody is ipilimumab.

7. A method for the treatment of Hepatitis B virus (HBV) infection, said method comprising administering to a subject in need thereof a combination attenuated non-pathogenic avian double-stranded (ds) RNA viral vector (IBDV) with an anti-PD-1 antibody and/or an anti-CTLA-4 antibody.

8. The method of claim 7, wherein the viral vector is R903/78.

9. The method of claim 7, said method comprising administering a dose of 0.5 mg/kg or lower of the anti-PD-1 antibody and a dose of 0.3 mg/kg or lower of the anti-CTLA-4 antibody.

10. The method of claim 7, wherein the anti-PD-1 antibody and the anti-CTLA-4 antibody are administered simultaneously or sequentially.

11. The method of claim 7, wherein the anti-PD-1 antibody and the anti-CTLA-4 antibody are administered at different times.

12. The method of claim 7, wherein the anti-PD-1 antibody is nivolumab or pembrolizumab and the anti-CTLA-4 antibody is ipilimumab.
